(19) United States
(12) Patent Application Publication
(10) Pub. No.: US 2015/0247168 A1
(43) Pub. Date: Sep. 3, 2015

(54) Use of Natural Biocide in the Process of Ethanol Production from Various Sources

(71) Applicant: Jadyr Mendes De Oliveira, (US)

(72) Inventors: Jadyr Mendes De Oliveira, Sao Paolo (BR); Rafael De Araújo Borges, Sao Paulo (BR); Mauricio Sergio Esteller, Sao Paulo (BR)

(21) Appl. No.: 14/391,220

(22) PCT Filed: Dec. 10, 2012
(86) PCT No.: PCT/BR2012/000509

§ 371 Date: Oct. 8, 2014

(30) Foreign Application Priority Data
Nov. 23, 2012 (BR) BR1020120298392

(51) Int. Cl. C12P 7/14

(52) U.S. Cl. CPC C12P 7/14

(57) ABSTRACT

Use of a biocide mixture containing natural biological ingredients for microbial controlling during the process of alcoholic fermentation of starch sources and/or other fermentable sugars from sugarcane, corn, sorghum, wheat, barley, potato, cassava, rice, malt, grapes, juices and several fruits, wherein lignocellulosic sources, such as leaves, wood, bagasse, bran, grass, husks, seeds can also be used, followed by chemical and/or enzymatic treatment, for producing first and second generations fuel ethanol.

The use of natural biocide in the process of ethanol production from various sources herein proposed, also relates to fermentation processes for the production of ethanol for human consumption, such as alcohol from cereals and corn, as well as fermented and/or distilled beverages such as beer, wine and cachaça.

The present invention refers to the use of natural biocide during the process of ethanol production from various sources, and more specifically, to the use of a biocide mixture containing natural biological ingredients, which helps in microbial controlling during the process of alcoholic fermentation of starch sources and/or other sugars from sugarcane, corn, sorghum, wheat, barley, potato, cassava, rice, malt, grapes, juices and several fruits, wherein lignocellulosic sources, such as leaves, wood, bagasse, bran, grass, husks, seeds can also be used, followed by chemical and/or enzymatic treatment, for producing first and second generations fuel ethanol.

The use of natural biocide in the process of ethanol production from various sources herein proposed, also relates to fermentation processes for the production of ethanol for human consumption, such as alcohol from cereals and corn, as well as fermented and/or distilled beverages, such as beer, wine and cachaça.

Alcoholic fermentation is a process performed by several organisms, including Saccharomyces cerevisiae, Saccharomyces uvarum, Saccharomyces carlsbergensis, Saccharomyces chevalieri, Candida krusei, Candida guilliermondii Candida tropicalis, Candida diddensiae, Candida fabianii, Candida intermedia, Candida maltosa, Candida santamariae, Candida colliculosa, Pichia membranaefaciens, Cryptococcus kuetzingii, Hansenula polymorpha, Kloeckera corticis, Rhodotorula pallida, Rhodotorula rubra, Rhodotorula minuta, Torulopsis norvegica and Trichosporon cutaneum.

In the fermentation process, the must contains nutrients (sucrose, glucose, amino acids, minerals, vitamins, folic acid, etc.) and favorable conditions (temperature, pH, oxygen amount) to bacterial development.

Therefore, it is necessary to control the bacterial growth, without interfering in process for producing ethanol or its products, since contaminating bacteria, usually Gram-positive ones, acidify the medium mostly via lactic, butyric, acetic acid fermentation and other fermentation that may occur alone or concomitantly, and inhibit yeast action, caused by products from their metabolism, or due to the drastic reduction of nutrients amount, affecting, thus, the quality and yield of the produced ethanol.

Acetic fermentation is attributed mainly to species Acetobacter aceti, A. pasteurianum, A. acetosum, A. kuntzegianum and A. suboxydans.

Lactic fermentation is attributed mainly to species Lactobacillus acidophilus, L. bulgaricus, L. casei and L. leischmanii e Streptococcus lactis.

Butyric fermentation is attributed mainly to species Clostridium pasteurianum and C. saccharobutyricum.

Dextran fermentation is attributed mainly to species Leuconostoc mesenteróides.

Levan fermentation is attributed mainly to species from genera Bacillus, Aerobacter e Streptococcus.

The fuel ethanol fermentation steps from sugarcane are:

milling (in order to obtain juice);

juice treatment (lime, sulphite, H₂SO₄);

pre-evaporation; must preparation;

fermentation;

centrifugation, and

distillation.

The fermentation process is not aseptic, and thus, it is very susceptible to microbial contamination.

The production of ethanol from starch occurs in several steps; at first grains milling (starch) occurs, which may be performed in a dry or wet manner; then, water is added to make the reaction medium more appropriate, and alpha-amylase enzymes are added (which may be from different strains) so that starch is liquefied; following this step the saccharification occurs, during which a must with high glucose concentrations is intended to be obtained. To that conversion glucoamylases are used, and thereafter must fermentation occurs, and in that step, there shall be attention to microbial contamination, which may interfere in the final product quality/quantity and process efficiency, and then ethanol is finally obtained by means of distillation processes.

The cellulosic ethanol comes from various sources, which may be divided in six categories:

1—Harvesting waste (cane bagasse, corn stover, wheat straw, rice husk, barley straw, barley husk, sorghum bagasse, olive seed and olive pulp);

2—Hardwood: aspen, poplar;

3—Softwood: pine tree, spruce;

4—Cellulose waste: newspaper, stationery and recycled paper;

5—Herb biomass: alfafa, hay, and several types of grass; and

6—Solid urban waste.

Currently in Brazil, the most abundant raw material is harvesting waste, more specifically sugarcane bagasse, composed of lignin (20 to 30%), cellulose (40 to 45%) and hemicellulose (30 to 35%).

The production of cellulosic ethanol comprises five main steps: biomass pretreatment; cellulose hydrolysis; hexoses fermentation; separation and effluents treatment.

Biomass pretreatment may be performed in several ways: using acids (hydrochloric, sulfuric, nitric); hydroxides (sodium hydroxide, calcium hydroxide); thermal treatment; wet oxidation using organic solvents, enzymes; or combined use of those methods.

The solid part, produced in the pretreatment, contains cellulose, and the liquid fraction contains hemicellulose hydrolysate. This solid part is then hydrolysed by enzymes or acids, the juice is fermented and the sugars from cellulose hydrolysis are transformed in alcohol.

Another method of cellulosic ethanol production comprises the same steps as the above described process, however, saccharification and fermentation occurs simultaneously, in order to improve the process yield and reduce costs with equipment maintenance. This process is named Simultaneous Saccharification and Fermentation (SSF).

In both described processes, the conversion of cellulose to glucose takes 12 to 96 hours. This time provides contaminating bacteria growth, which consume the produced glucose, reducing the yield and causing problems in the subsequent fermentation. Therefore, the use of an effective antimicrobial agent against bacteria and inert to yeast is required. The antimicrobial shall also be resistant to inhibitors generated during lignocellulosic raw material pretreatment (furfural, hydroxymethilfurfural, short chain acids).

The biocide may also be applied to ethanol for human consumption. The most consumed alcoholic beverage in Brazil is beer. In the beer production, it is required some steps which aim the production of a must rich in sugars and amino acids, which shall be fermented. Beer producing steps are:

malt milling;

mashing;

boiling;

fermentation;

maturation;

filtration, and

packanging.

Sugarcane fermentation steps for cachaça production are:

milling (in order to obtain juice);

juice treatment (lime, sulphite, H₂SO₄);

pre-evaporation;

must preparation;

fermentation;

centrifugation, and

distillation.

The fermentation process is not aseptic, and thus, it is very susceptible to microbial contamination.

Contamination control in ethanol plants both from cane and starch may be performed with the use of synthetic antibiotics; however, there is a need for the non-use of those antibiotics, as they leave traces in the byproducts used in animal and human feeding, or used as fertilizer in plantations, which are a byproduct with high added value.

The main byproduct of sugar-alcohol industry (cane) is yeast. The restriction to the use of antibiotics is valid, as those dry yeasts, yeast extracts and other products derived from yeast are used in animal feeding and in some cases in human feeding.

The main byproduct of starch ethanol industry is a solid waste which is used for animal feeding, therefore, it must also be free from antibiotics.

Another advantage of the natural biocide when compared to synthetic antibiotics is that the bacteria do not develop resistance to the compound, so it may be used constantly with no loss of action.

The Brazilian application P10705512-9 A2 claims, in addition to the process of nisin production, the use of nisin together with EDTA, lysozyme and Tween 80, in the steps of cane milling and must fermentation.

The present invention differs from the previously mentioned P10705512-9 A2, since the present invention considers, in addition to the interactions of the mentioned ingredients, the dosage points, as the biocide of the invention may require extreme conditions for action (sugars concentration, presence of inhibitors in the raw material, pH, temperature, total solids concentration and amount of water in the fermentation medium).

The biocide is intended to control the proliferation of harmful microorganisms in the fermentation process, either of cane, starch and other sugars.

The biocide is composed mostly of polylysine (10 to 100%), and may contain additives lysozyme (1 to 10%), glucose oxidase (1 to 10%), hop alpha and beta acids (1 to 20%), sodium sulphite (10 to 90%), EDTA (up to 90%) and nisin (1 to 10%), depending on the process to be controlled.

The biocide main action is against Gram positive bacteria, but it also acts on Gram negative bacteria.

The biocide is resistant to acid (pH 2.0) and heating (105° C.), as in the fermentation process those parameters are important.

In cane mills, optimum dosage points for the biocide of the present invention are:

1. in mill water (5 to 80 ppm concerning the water volume);

2. in decantation tank (5 to 80 ppm concerning the juice);

3. in the fermentation—fermentation tub (5 to 80 ppm concerning the juice), and

4. in the ferment treatment vat before fermentation (5 to 80 ppm concerning the tub total volume).

The biocide may be dosed in isolated points (1 or 2 or 3 or 4), or in combined points (1 and 2; 1 and 3; 1 and 4; 2 and 3; 2 and 4; 3 and 4; 1, 2 and 3; 1, 2 and 4; 1, 3 and 4; 2, 3 and 4; 1, 2, 3 and 4), with lower doses along the fermentation (1 to 40 ppm), to keep contamination low.

In the starch sources fermentation process (corn, sorghum, wheat, barley, potato, cassava, rice, malt, grapes, juices and several fruits), the biocide may be dosed:

1. in liquefaction (5 to 80 ppm), and

2. in fermentation tub must (5 to 80 ppm), with lower doses along the fermentation (1 to 40 ppm) to keep contamination low.

The biocide may be dosed in isolated points (1 or 2) or in combined points (1 and 2).

In lignocellulosic sources fermentation process, the biocide may be dosed:

1. during cellulose, hernicellulose, lignin hydrolysis (5 to 100 ppm), and

2. during fermentation (5 to 100 ppm).

The biocide may be dosed in isolated points (1 or 2) or in combined points (1 and 2), when a simultaneous process of saccharification and fermentation occur.

In beer production, the biocide may be dosed:

1. in mashing (5 to 80 ppm);

2. in fermentation (5 to 80 ppm), and

3. in maturation (5 to 80 ppm).

The biocide may be dosed in isolated points (1 or 2 or 3) or in combined points (1, 2 and 3; 1 and 3; 2 and 3).

In cachaça production, the steps are similar to those in sugarcane fermentation. The dosage points are:

1. in mill water;

2. in the decantation tank, and

3. in the fermentation tub.

The biocide may be dosed in isolated points (1, 2 or 3), or in combined points (1, 2 and 3; 1 and 3; 2 and 3).

Some industrial tests were performed to demonstrate the efficacy of those bacteriocynes in sugarcane mills, and the results show fast reduction of the bacterial population, among the effects, the following decrease may be mentioned: 48.46% for Lactobacillus fermentum; 84.38% for L. plantarum; 93.86% for L. casei; 77.21% for Bacillus subtilis, and 55.11% for Acetobacter pasteurianus.

1. USE OF NATURAL BIOCIDE IN THE PROCESS OF ETHANOL PRODUCTION FROM VARIOUS SOURCES to control the proliferation of harmful microorganisms to the fermentation process of cane, starch and other sugars, wherein the biocide is composed mostly of polylysine (10 to 100%), and may contain additives lysozyme (1 to 10%), glucose oxidase (1 to 10%), hop alpha and beta acids (1 to 20%), sodium sulphite (10 to 90%), EDTA (up to 90%) and nisin (1 to 10%), depending on the process to be controlled.

2. USE OF NATURAL BIOCIDE IN THE PROCESS OF ETHANOL PRODUCTION FROM VARIOUS SOURCES, according to claim 1, wherein the biocide main action is against Gram positive bacteria.

3. USE OF NATURAL BIOCIDE IN THE PROCESS OF ETHANOL PRODUCTION FROM VARIOUS SOURCES, according to claim 1, wherein the biocide may also act against Gram negative bacteria.

4. USE OF NATURAL BIOCIDE IN THE PROCESS OF ETHANOL PRODUCTION FROM VARIOUS SOURCES, according to claim 1, wherein the biocide is resistant to acid (pH 2.0) and heating (up to 121° C.).

5. USE OF NATURAL BIOCIDE IN THE PROCESS OF ETHANOL PRODUCTION FROM VARIOUS SOURCES, according to claim 1, wherein in cane mills, the biocide may be dosed: (1) in mill water (5 to 80 ppm concerning the water volume); (2) in decantation tank (5 to 80 ppm concerning the juice); (3) in the fermentator—fermentation tub (5 to 80 ppm concerning the juice), and (4) in the ferment treatment vat before fermentation (5 to 80 ppm concerning the tub total volume).

6. USE OF NATURAL BIOCIDE IN THE PROCESS OF ETHANOL PRODUCTION FROM VARIOUS SOURCES, according to claim 5, wherein the biocide may be dosed in isolated points (1 or 2 or 3 or 4), or in combined points (1 and 2; 1 and 3; I and 4; 2 and 3; 2 and 4; 3 and 4; 1, 2 and 3; 1, 2 and 4; 1, 3 and 4; 2, 3 and 4; 1, 2, 3 and 4), with lower doses along the fermentation (1 to 40 ppm), to keep contamination low.

7. USE OF NATURAL BIOCIDE IN THE PROCESS OF ETHANOL PRODUCTION FROM VARIOUS SOURCES, according to claim 1, wherein in the starch sources fermentation process, the biocide may be dosed: (1) in liquefaction (5 to 80 ppm), and (2) in fermentation tub must (5 to 80 ppm), with lower doses along the fermentation (1 to 40 ppm) to keep contamination low.

8. USE OF NATURAL BIOCIDE IN THE PROCESS OF ETHANOL PRODUCTION FROM VARIOUS SOURCES, according to claim 7, wherein the biocide may be dosed in isolated points (1 or 2) or in combined points (1 and 2).

9. USE OF NATURAL BIOCIDE IN THE PROCESS OF ETHANOL PRODUCTION FROM VARIOUS SOURCES, according to claim 1, wherein in lignocellulosic sources fermentation process, the biocide may be dosed: (1) during cellulose, hemicellulose, lignin hydrolysis (5 to 100 ppm), and (2) during fermentation (5 to 100 ppm).

10. USE OF NATURAL BIOCIDE IN THE PROCESS OF ETHANOL PRODUCTION FROM VARIOUS SOURCES, according to claim 9, wherein the biocide may be dosed in isolated points (1 or 2) or in combined points (1 and 2), when a simultaneous process of saccharification and fermentation occur.

11. USE OF NATURAL BIOCIDE IN THE PROCESS OF ETHANOL PRODUCTION FROM VARIOUS SOURCES, according to claim 1, wherein in beer production, the biocide may be dosed: (1) in mashing (5 to 80 ppm); (2) in fermentation (5 to 80 ppm), and (3) in maturation (5 to 80 ppm).

12. USE OF NATURAL BIOCIDE IN THE PROCESS OF ETHANOL PRODUCTION FROM VARIOUS SOURCES, according to claim 11, wherein the biocide may be dosed in isolated points (1 or 2 or 3) or in combined points (1, 2 and 3; 1 and 3; 2 and 3).

13. USE OF NATURAL BIOCIDE IN THE PROCESS OF ETHANOL PRODUCTION FROM VARIOUS SOURCES, according to claim 1, wherein in cachaça production, the dosage points are: (1) in mill water; (2) in the decantation tank, and (3) in the fermentation tub.

14. USE OF NATURAL BIOCIDE IN THE PROCESS OF ETHANOL PRODUCTION FROM VARIOUS SOURCES, according to claim 13, wherein the biocide may be dosed in isolated points (1, 2 or 3), or in combined points (1, 2 and 3; 1 and 3; 2 and 3).
